(19) United States
(12) Patent Application Publication
(10) Pub. No.: US 2025/0312198 A1
(43) Pub. Date: Oct. 9, 2025

(54) AMBIENT NOISE REDUCING HEADSET WITH INTERCHANGEABLE PADDING AND NOISE REDUCING COVERS

(71) Applicant: Atlantic Signal, LLC, Topeka, KS (US)

(72) Inventor: Mads Staerke, Frederiksberg C (DK)

(73) Assignee: Atlantic Signal, LLC, Topeka, KS (US)

(21) Appl. No.: 19/174,620

(22) Filed: Apr. 9, 2025

Related U.S. Application Data
(60) Provisional application No. 63/631,868, filed on Apr. 9, 2024.

(51) Int. Cl. A61F 11/14

(52) U.S. Cl. CPC A61F 11/14

(57) ABSTRACT

An ambient noise reducing headset with interchangeable padding and noise reducing covers provides a modular and configurable headset system, allowing a wearer to select an ear pad and/or ear cover for each of the left and right ears of the headset, or a single ear pad and ear cover in the case of a single-sided headset, to provide a desired noise attenuation and fit/comfort for each ear. The various ear pads and ear covers are configured to removably attach to a housing of the headset so that a wearer may swap out ear pads and ear covers as desired. The modular nature of the claimed headset thus allows a user to maintain and carry a single headset and housing assembly and to install a desired ear pad and ear cover as needed.

### CROSS REFERENCE TO RELATED APPLICATION

This application claims the benefit of U.S. Provisional Patent Application No. 63/631,868 filed Apr. 9, 2024, the disclosure of which is hereby incorporated herein in its entirety by reference.

### BACKGROUND OF THE INVENTION

### Field of the Invention

This invention relates generally to noise reduction apparatus, and more specifically to headsets for noise reduction.

### Background

Noise reducing headsets are used in numerous applications to reduce the intensity or level of ambient sound reaching the ear (or ears) of a wearer. As used herein, ambient sound is environmental or background noise that may be disruptive, dangerous, and/or uncomfortable to a wearer. Reduction of the sound level reaching the ear can provide comfort or convenience to the wearer, e.g., a reduced sound level may allow the wearer to better concentrate on a task or communicate with others, and/or a reduced sound level can also protect the wearer from hearing damage caused by dangerously loud ambient sound levels.

Depending on the environment in which a headset is being used, the sound-reducing and sound-absorbing qualities of an appropriate headset for that environment will vary. For example, in a very noisy environment, such as in proximity to a running aircraft engine, a user will typically want a headset having strong sound level attenuation properties, while in a less noisy environment, such as a call center with multiple people talking in the background, a user will typically want a headset having low or moderate sound level attenuation properties.

In order to meet the needs of users encountering various noise environments, manufacturers produce multiple versions of noise reducing headsets to allow users to choose the appropriate headset for their particular need, with various versions of headsets offering varying noise reduction and/or various sizes of padding and earcups to accommodate users' preferences. While that conventional approach meets the needs of many users who will simply select a single headset that best works in the environment the user typically encounters, other users who typically encounter multiple noise environments must buy and keep several headsets available so that they will have an appropriate headset as their environment changes. For example, communications headsets used by law enforcement agents often have noise reducing ear covers to allow the wearer to receive radio communication through a transducer in a covered ear, while still allowing some ambient sound. Other communications headsets may have a single ear cover, leaving one ear uncovered to allow the wearer to directly hear ambient sounds through the uncovered ear. Depending on the environment in which the headset is used, the user may need varying levels of sound reduction and/or different sound reduction in different ears, thus requiring the user to have multiple headsets available to meet the needs of a particular environment or to meet the needs of a changing environment.

Thus, it can be shown that there remains a need in the art for an improved ambient noise reducing headset that overcomes the shortcomings of known headsets.

### SUMMARY

Embodiments of the invention are defined by the claims below, not this summary. A high-level overview of various aspects of the invention is provided here to introduce a selection of concepts that are further described in the detailed description section below. This summary is not intended to identify key features or essential features of the claimed subject matter, nor is it intended to be used in isolation to determine the scope of the claimed subject matter. In brief, this disclosure describes, among other things, an ambient noise reducing headset with interchangeable padding and noise reducing covers.

In one embodiment, the headset comprises a housing for receiving and removably attaching a desired ear pad, ear cover, and/or a combination ear pad/cover. The housing is attached to a headband configured to extend around a portion of a head of a wearer and may be configured to snugly fit across the top of a wearer's head. With the headband in place on the wearer's head, the housing is positioned adjacent to and spaced a distance from the wearer's ear such that the attached ear pad is positioned to cover at least a portion of the wearer's ear. The housing and the attached ear pad form an earcup configured to rest on or around the wearer's ear and in covering relationship therewith. Because the ear pad is removably or releasably attached to the housing, the wearer may select any one of several ear pads and/or ear covers that are interchangeably couplable to the housing to provide a level of noise attenuation associated with the selected ear pad and/or ear cover as desired or as dictated by ambient noise conditions. For example, in a less noisy environment the wearer may select an ear pad that allows ambient noise to reach the wearer's ear, while in a more noisy environment the wearer may select an ear pad that attenuates most of the ambient noise, allowing very little of that noise to reach the wearer's ear. The headband is configured to position the housing such that the earcup formed by the selected ear pad and/or ear cover attached to the housing is positioned relative to the wearer's ear to provide the level of noise attenuation associated with the selected ear pad and/or ear cover.

In one aspect, the noise attenuation provided by the selected ear pad and/or ear cover (or the combination) is dependent upon various factors and parameters. For example, the type and amount of sound absorbing material within the ear pad may be varied to adjust the amount of noise attenuation, and the type of padding and sound absorbing material used may be varied to affect the fit and comfort of the ear pad. For example, some ear pads may cover the entire ear while others may be configured to rest upon the ear or to cover only a portion of the ear.

Thus, the sound attenuation and fit/comfort of each ear pad can be configured as required or desired by a wearer. Similarly, the material and configuration of an ear cover (typically an outer shell or outer surface affixed to the ear pad) may be selected to provide desired noise attenuation or other characteristics. For example, a hard plastic ear cover may provide a low level of sound attenuation but a high level of physical protection to the ear, e.g., for use in a hazardous environment. Conversely, a soft ear cover comprising sound absorbing material may provide strong noise attenuation, especially when used in conjunction with a noise attenuating ear pad. Depending on the needs of the wearer and the environment in which they will be using the headset, the wearer can thus select any one of multiple ear pads/ear cover combinations for attachment to the housing.

In another embodiment, the headset includes two housings attached to opposite sides of the headband such that a wearer may attach two similar or dissimilar ear pads to provide a desired combination of sound attenuation. For example, a wearer may use a high attenuation ear pad to cover the left ear while using a lesser attenuating ear pad on the right ear to allow some level of ambient noise. As described relative to the headset with one housing, the headband positions the two housings coupled thereto spaced a distance from the respective ear of the wearer to provide the level of noise attenuation associated with each ear pad and/or cover coupled to the two housings. The distance that each of the housings is spaced from the respective ear may be similar or different, and the distance may be dependent on the ear pad and/or ear cover attached thereto.

Thus, the claimed invention provides for a modular and configurable noise reducing headset, allowing a wearer to select an ear pad and/or ear cover for each of the left and right ears of the headset (or a single ear pad/ear cover in the case of a single-sided headset) to provide the desired noise attenuation and fit/comfort for each ear. The various ear pads and ear covers are configured to removably attach to the corresponding housing so that a wearer may swap out ear pads/ear covers as desired. The modular nature of the claimed headset thus allows a user to maintain and carry a single headset assembly and to swap out the ear pads/ear covers as desired.

### DESCRIPTION OF THE DRAWINGS

Illustrative embodiments of the invention are described in detail below with reference to the attached drawing figures, and wherein:

FIG. 1 is an elevation view of a headset with a housing for accommodating a desired cover and/or ear padding in accordance with an exemplary embodiment of the present invention.

FIG. 2 is a front perspective view of the headset with the housing of FIG. 1.

FIG. 3 is a rear perspective view of the headset with the housing of FIG. 1.

FIGS. 4 through 7 depict the headset with the housing of FIG. 1, with a first embodiment of an exemplary ear pad, which is attached to the housing. FIG. 4 shows the headset secured on a wearer's head with the ear pad and housing positioned relative to an ear of the wearer.

FIGS. 8 through 10 depict the headset with the housing of FIG. 1, with a second embodiment of an exemplary ear pad with cover, which are attached to the housing.

FIGS. 11 through 13 depict the headset with the housing of FIG. 1, with a third embodiment of an exemplary ear pad, which is attached to the housing.

FIGS. 14 and 15 depict the headset with the housing of FIG. 1, with a fourth embodiment of an exemplary ear pad with cover, which are attached to the housing.

FIGS. 16 and 17 depict the headset with the housing of FIG. 1, with a fifth embodiment of an exemplary ear pad with cover, which are attached to the housing.

FIG. 18 depicts the headset with the housing of FIG. 1, with a sixth embodiment of an exemplary ear pad with cover, which are attached to the housing.

FIGS. 19 and 20 depict a headset similar to FIG. 1 but configured for covering both ears of a wearer, with a seventh embodiment of an exemplary ear pad attached to each of first and second housings of the headset.

### DETAILED DESCRIPTION

The subject matter of select embodiments of the invention is described with specificity herein to meet statutory requirements. But the description itself is not intended to necessarily limit the scope of claims. Rather, the claimed subject matter might be embodied in other ways to include different components, steps, or combinations thereof similar to the ones described in this document, in conjunction with other present or future technologies. Terms should not be interpreted as implying any particular order among or between various steps herein disclosed unless and except when the order of individual steps is explicitly described. The terms “about” or “approximately” as used herein denote deviations from the exact value in the form of changes or deviations that are insignificant to the function. Further, the terms “outward” or “outer”, and variations thereof, are used to mean a side or portion of a component facing away from a user or wearer or facing in a direction away from the component.

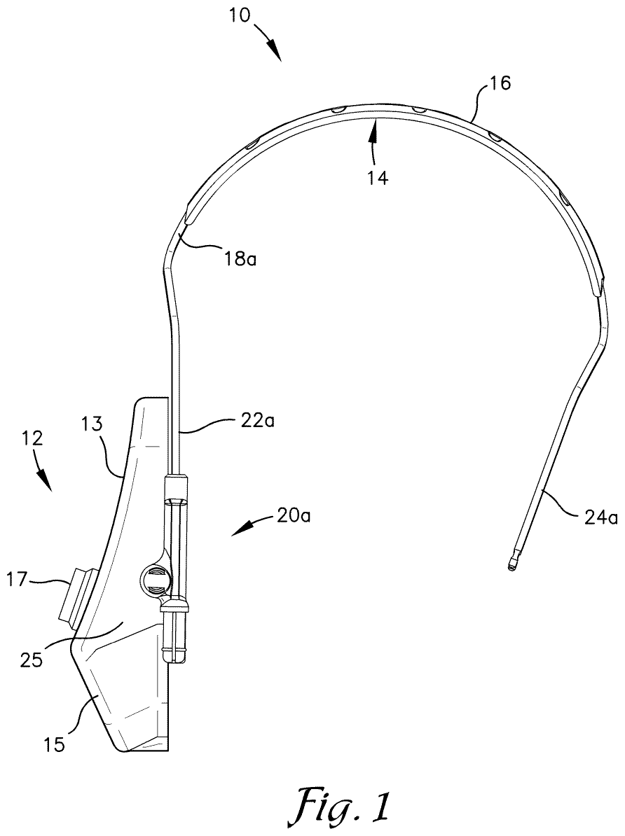

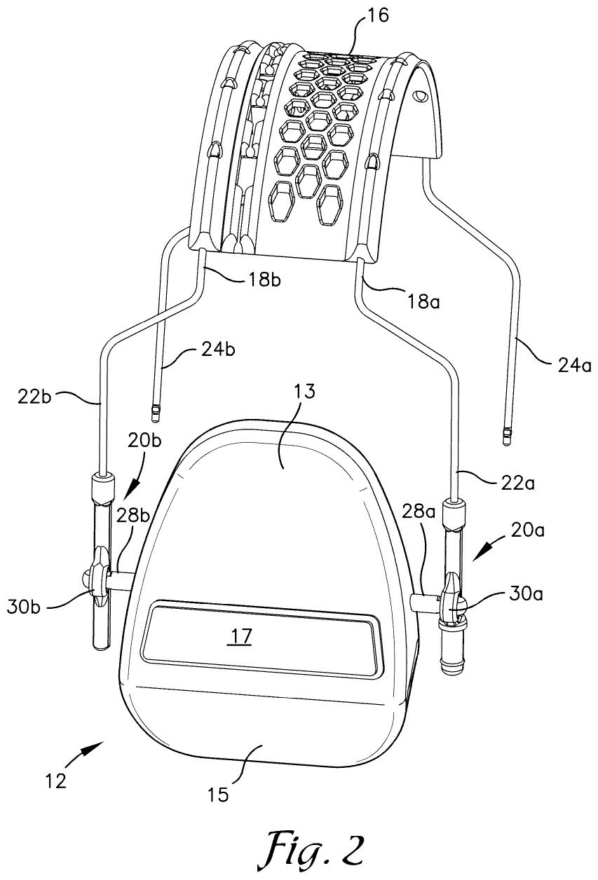

Looking first to FIGS. 1, 2, and 3, an ambient noise reducing headset configured for coupling thereto an interchangeable ear padding and/or noise reducing ear cover in accordance with an exemplary embodiment is depicted generally by the numeral 10. The headset 10 generally includes a housing 12 for receiving and removably attaching an ear pad, which may include an ear cover, selected from a plurality of sized and shaped ear pads, and a headband 14 coupled to the housing 12 for attachment of the headset 10 to the head of a wearer such that the housing 12 (and any attached ear pad and/or ear cover) is positioned over the ear of a wearer so that the ear pad and/or ear cover provides noise attenuation to reduce the level of ambient noise allowed into the wearer's ear.

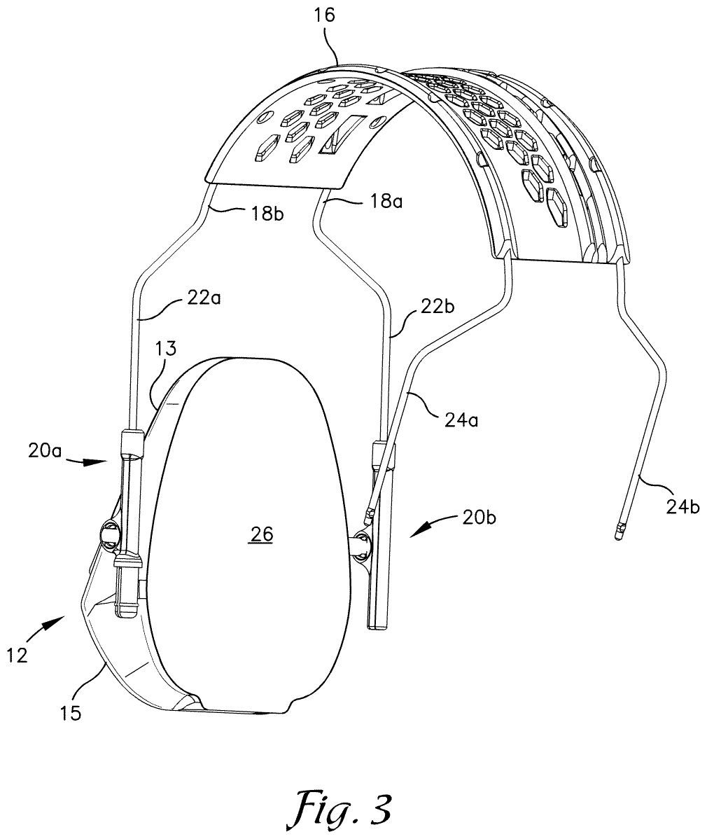

In the embodiment shown, the housing 12 includes a sloped upper portion 13 generally shaped to conform to the upper ear of the wearer and an oppositely sloped lower portion 15 generally shaped to conform to the lower ear of the wearer. The upper portion 13 and the lower portion 15 form an outside surface of the housing 12. The upper portion 13 and lower portion 15, in conjunction with a side wall 25 and a surface 26, may define an interior of the housing 12 in which electronics and/or noise attenuation material, as described herein, may be contained. In an embodiment shown in FIG. 3, the generally oval shaped interior surface 26 of the housing 12 is configured to be spaced apart from and in covering relationship with a wearer's entire ear. In an embodiment, the interior surface 26 may be an inner cover or screen that is flat and configured to protect components or material within the housing 12.

A raised control pad portion 17 protruding outwardly from the upper portion 13 provides a surface for mounting one or more switches, lights, or other controls for electronics that may be contained in the housing 12 of the headset 10 or within an ear pad removably secured to the housing 12 in some embodiments. The controls of the control pad 17 are directed outward from the housing 12 and are configured to be accessible by a wearer wearing the headset 10. It is to be understood that the housing 12 shown is simply intended to provide an example of a housing 12 with which the removable and interchangeable ear pads, ear covers, and/or combination ear pads with ear covers could be utilized.

Headband 14 comprises a wide, semi-circular or arch-shaped band portion 16 that extends between opposite ends thereof to extend around a portion of a wearer's head, and in an embodiment, to conform generally to the contour of an upper portion of a wearer's head. In an embodiment, the headband 14 may be configured to conform generally to a back of a wearer's head or neck. The band includes a plurality of ventilation holes formed therethrough, with channels formed along opposite sides of the band 16 to receive first and second support rods 18a and 18b, which are similarly arch-shaped and extend past each end of the band 16 to form the headband 14. In an embodiment, the first and second support rods 18a and 18b extend in parallel and provide a frame to support the band 16. In an embodiment, the headband 14 is adjustable to conform to the head of a wearer.

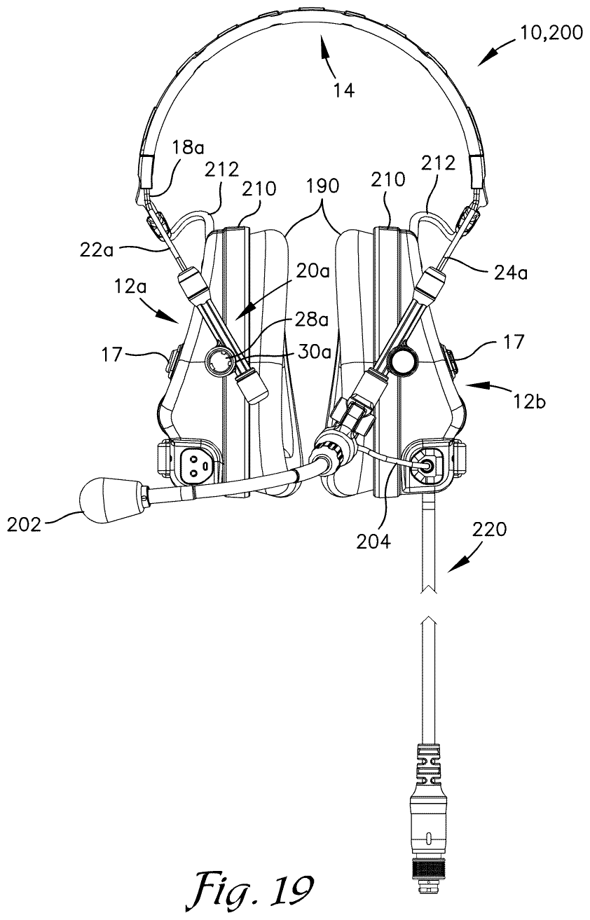

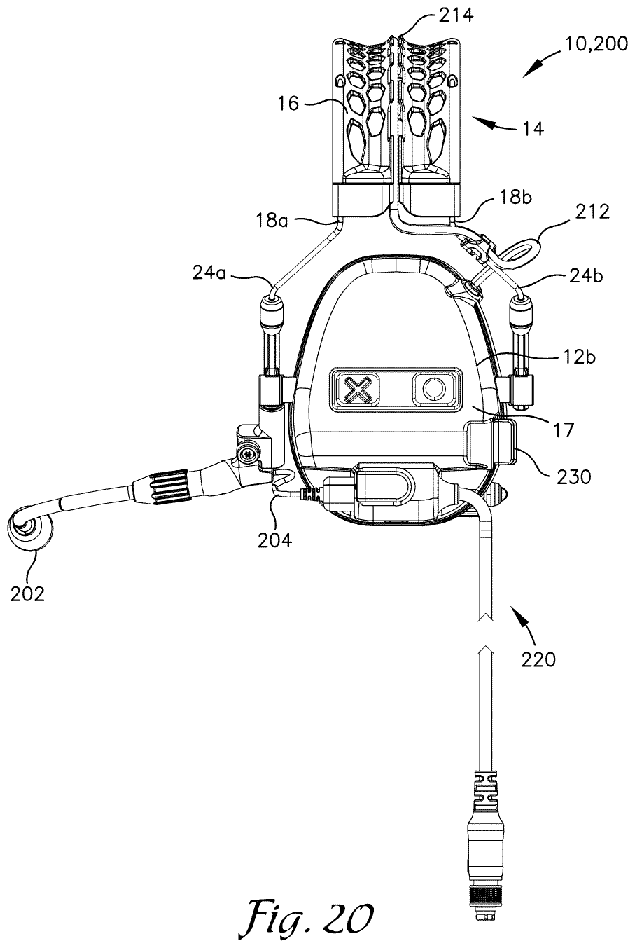

The ends of each the first and second support rods 18a and 18b are attached to or form first tines 22a and 24a and second tines 22b and 24b, respectively. The first and second tines 22a and 22b and the first and second tines 24a and 24b flare outwardly or laterally away from each other to form a space between each set of first and second tines to accommodate the corresponding housing 12 therebetween. The tines 22a, 22b, 24a, and 24b may also be angled inward or toward a head of a wearer to position the housing 12 relative to an ear of a wearer. The tines 22a, 22b, 24a, and 24b may also be referred to as arm members or couplers. Thus, in the exemplary embodiment of FIGS. 1 through 3, the housing 12 is generally positioned and supported between first and second tines 22a and 22b, which form a first housing yoke, with the space between first and second tines 24a and 24b forming a second housing yoke able to accommodate a second housing if desired (such as in a headset for covering both ears of the wearer as shown in FIGS. 19 and 20, described herein). The first and second tines 22a and 22b are inserted into respective first and second barrel connectors 20a and 20b that are attached to the housing 12 by corresponding first and second pins 28a and 28b extending from the side wall 25 of the housing 12. The pins 28a and 28b are rotatably coupled to respective first and second bores or bearing surfaces 30a and 30b of the first and second barrel connectors 20a and 20b, and the housing 12 is thus pivotably attached to the first and second tines 22a and 22b of the headband 14, allowing the housing 12 to rotate relative to and between the tines 22a and 22b to provide a snug and comfortable fit of the housing 12 in proximity to the wearer's head. In an embodiment, the tines 24a and 24b are similarly coupled to a second housing.

In the embodiment depicted in FIGS. 1 through 3, the headset 10 includes a first housing 12 provided to form a covering over a first ear of the wearer. In some embodiments a second housing will be provided and may be selectively attached to the opposite side of the headset such that the headset 10 is able to accommodate ear pads and/or ear covers for both ears of a wearer (see FIGS. 19 and 20) when desired. It should be further understood that in an embodiment, additional padding, covers, or another fitment piece coupled to one or both of the tines 22a and 22b and/or the tines 24a and 24b could be included to provide a comfortable fit of the headband 14 to the wearer.

Thus, as just described the headset 10 provides one or two housings 12 connected to a headband 14 that allows a wearer to position the one or two housings 12 adjacent to the wearer's ear or ears. As discussed above, and as will now be described in more detail, each housing 12 is configured to receive and removably attach thereto one of a plurality of ear pads, or ear pads that include ear covers, to allow the wearer to select an ear pad and/or ear cover for each housing to provide a desired level of noise attenuation, comfort, and fitment.

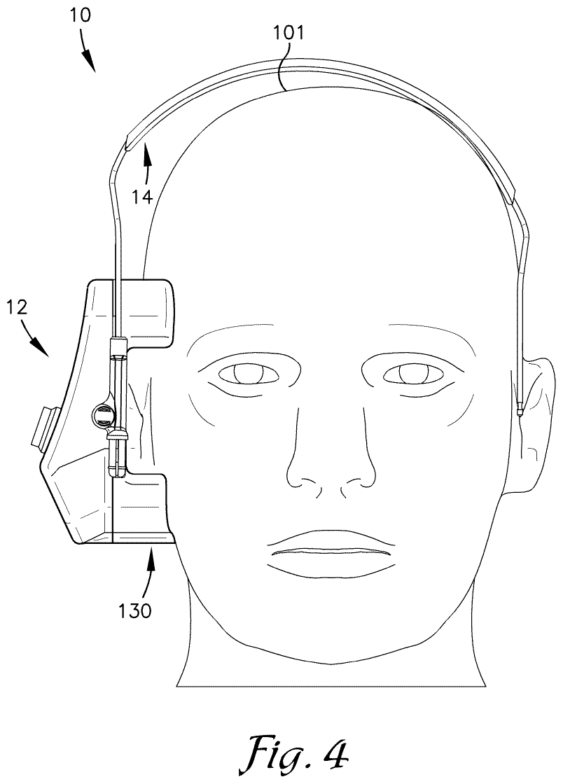

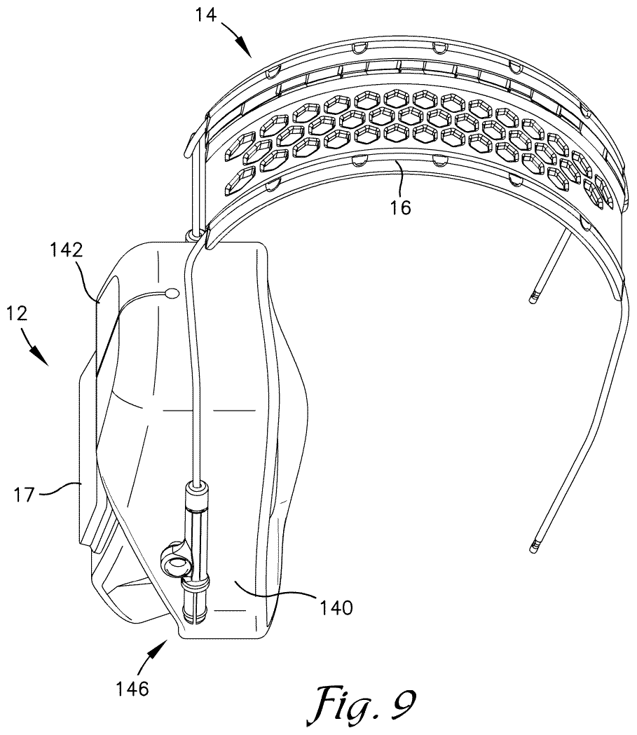

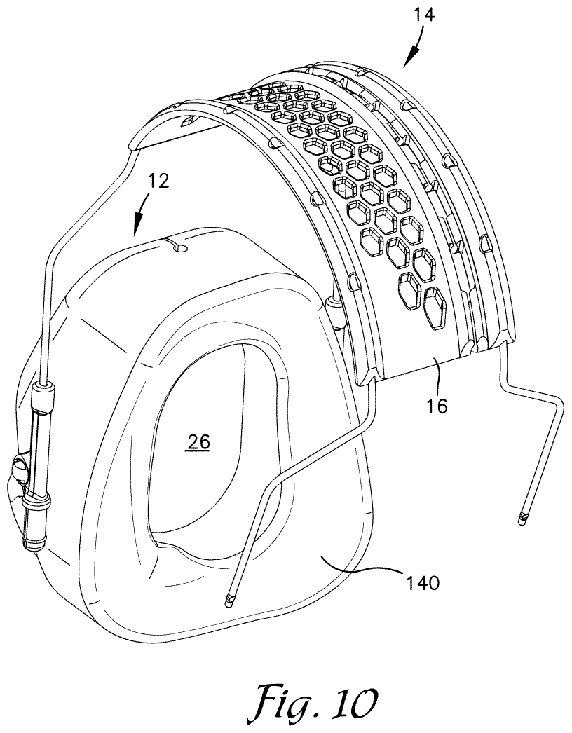

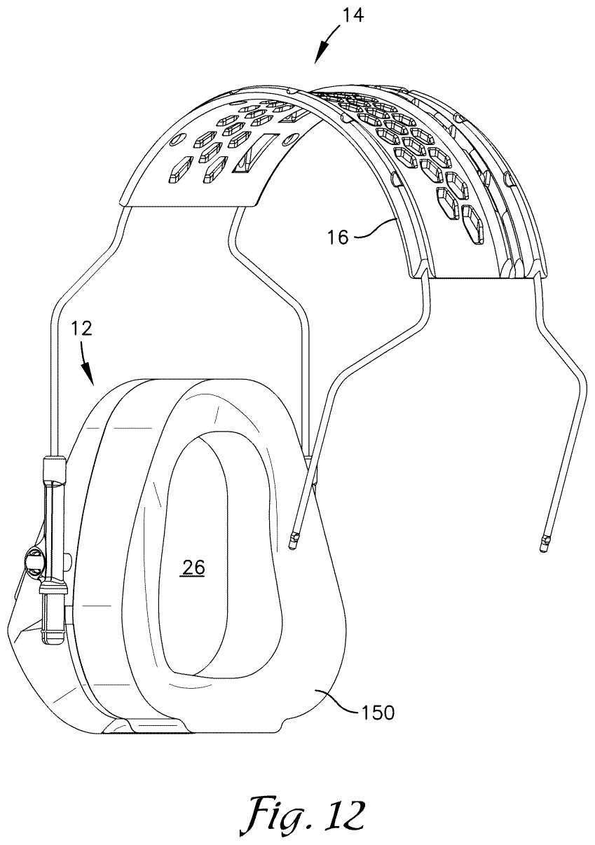

Looking to FIGS. 4 through 20, several variations of ear pads and/or ear covers for removably attaching to the at least one housing 12 of the headset 10 as described above are depicted. Each of the ear pads and/or ear covers is configured to removably attach to (or around) the corresponding housing so that a wearer can select a desired ear pad and/or ear cover with each ear pad and/or ear cover providing noise attenuation material and/or padding to achieve a desired noise level attenuation and comfort level. The ear pads are configured to be reuseable, but it is contemplated that the ear pads could be disposable. The ear pads and/or ear covers removably attached to the housing are configured to form earcups that may allow some ambient noise to enter an ear or may be circumaural earcups that surround an ear or rest around an outside of an ear or over-the-ear earcups that sit on an outer surface of an ear, either of which may completely enclose the ear and/or form a circumaural seal to minimize or eliminate ambient noise entering the ear. In an embodiment, an ear pad is not formed from a rigid material but rather the ear pad and/or ear cover have elastic qualities to enable the ear pad and/or ear cover to return to its original shape after being stretched or compressed, such as due to wearing or securing the housing to the ear pad and/or ear cover. In an embodiment, the ear pads and/or ear covers shown may be used with an audio device positioned within the housing and/or ear pad to allow the wearer to receive and/or send communications, while attaining the desired level of noise attenuation depending on the selected ear pad and/or ear cover. It should be understood that the various ear pads and/or ear covers depicted are exemplary and not limiting, and that other configurations of ear pads and/or ear covers may be used with the headset 10 that includes the headband 14 and at least one housing 12 of the present invention. Because each housing 12 can accept multiple and varied ear pads and/or ear covers, the claimed headset 10 provides a modular and configurable headset.

It should be further understood that there is overlap between the “ear pad” and “ear cover” as described herein, and that for any specific configuration the “ear pad” may blend into the “ear cover”. For example, the “ear pad” portion is generally the portion that contacts the wearer's head and thus fits around the wearer's ear and contacts the head to seal and prevent noise from entering the ear through that passageway. The “ear cover” portion is generally an outer shell or surface attached to, or integral with, the ear pad, that seals the ear pad and provides additional sound attenuation material. In some embodiments the ear cover may comprise a separate piece formed from a separate material that is attached to the ear pad. In some embodiments, the housing may act as the entire ear cover or a portion of the ear cover. In other embodiments, the ear pad and the ear cover may be of the same material, with the ear cover essentially an extension of the ear pad that may be formed integrally therewith. The term “ear pad” may be used to describe an ear pad or the ear pad that includes the ear cover, which will become evident in view of the embodiment being described.

Turning to FIGS. 4 through 7, a headset 10, which includes a headband 14 and housing 12 as in FIG. 1, is depicted with a first embodiment of an exemplary ear pad 130 attached to the housing 12. As shown in FIG. 4, the headband 14 attaches to the housing 12 as previously described, with the arched headband 14 extending upwardly around a wearer's head 101 to secure the headset 10 in place and position the housing 12 and ear pad 130 adjacent to the wearer's ear. The headset 10 with the ear pad 130 secured on the wearer's head 101 shown in FIG. 4 is exemplary of how the headset 10 and other embodiments of the ear pad described herein fit relative to a wearer's head 101.

In this embodiment, the ear pad 130 is attached to the housing 12, which in turn is attached to the headband 14. The ear pad 130 may be attached to the housing 12 by a releasable adhesive, by hook and loop fastener tabs, by an elastic band attached around the perimeter of the ear pad so that the ear pad is stretched over the housing, or by any other releasable attachment means known in the art.

As shown in the figures, the ear pad 130 includes gaps 132 in the pad material so that the pad 130 does not form a continuous or contiguous seal around the wearer's ear so that some ambient noise is allowed into the ear. In an embodiment, the ear pad 130 extends inward relative to the interior surface 26 of the housing 12 or toward the head 101 of the wearer. The ear pad 130 has a similar circumferential dimension to that of the housing 12, and the ear pad 130 and the housing 12 form an earcup that rests on the ear or rests around the outside of the ear. As described previously, the padding material of the ear pad 130 may be comprised of sound absorbing material to lessen the ambient noise level reaching the ear and may further be comprised of a comfortable padding material at the point(s) of contact of the pad with the wearer's head. Exemplary sound absorbing and/or padding materials may include compressible foams, gels, rubber or rubber-like materials including for example silicone rubber.

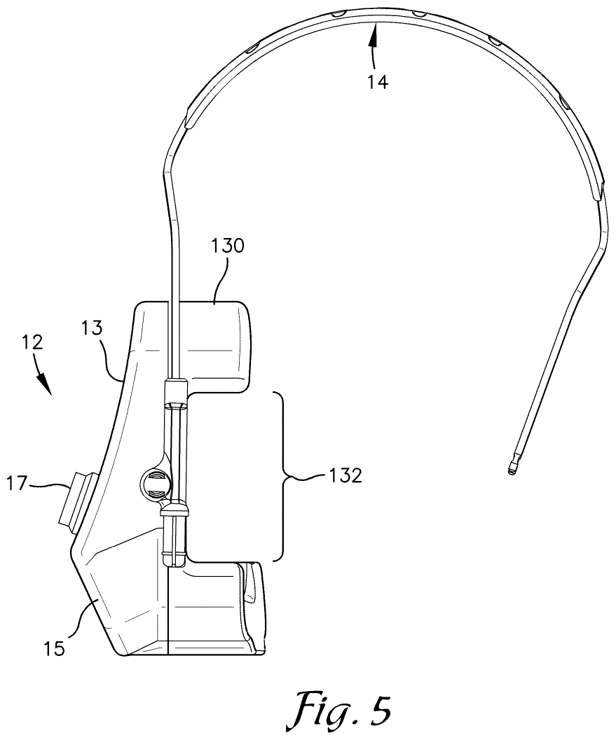

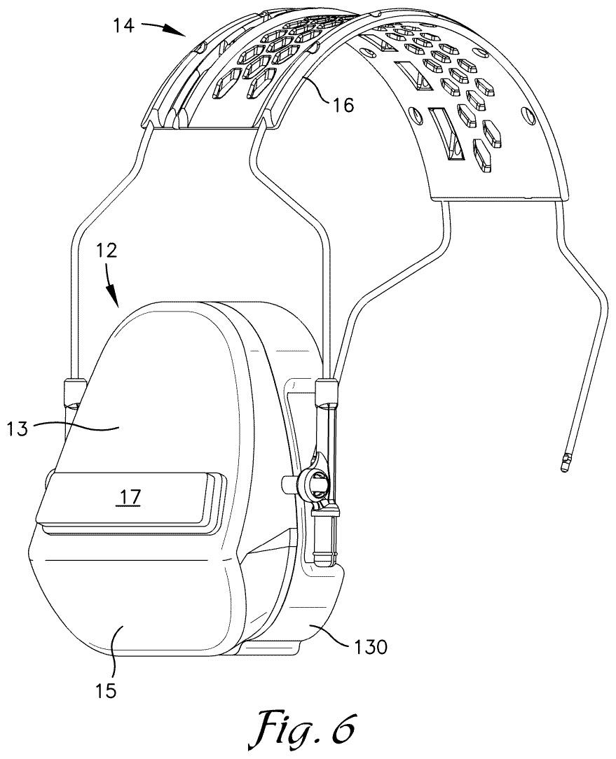

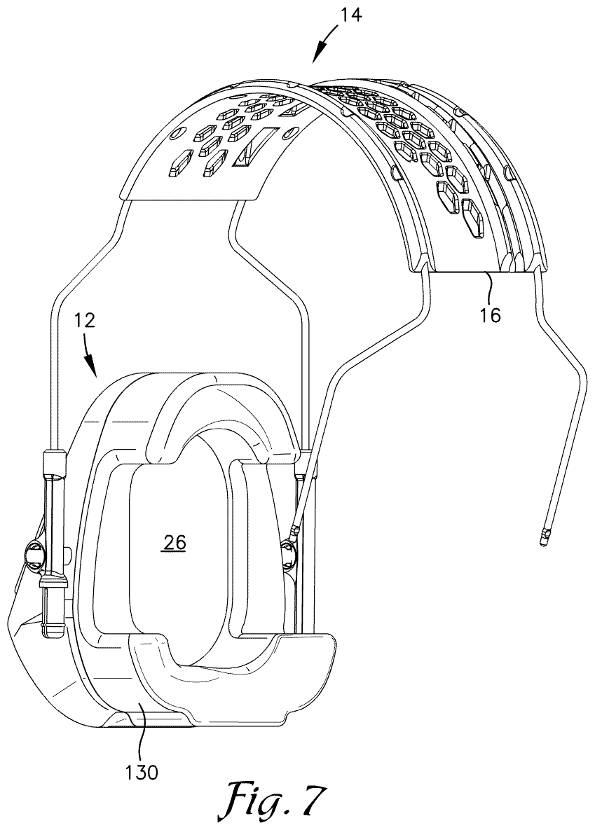

As best shown in FIGS. 6 and 7, in this embodiment the housing 12 acts as the ear covering, covering the outer portion of the ear pad 130 to prevent, diminish, or limit sound from directly entering the wearer's ear from that direction. As shown in FIGS. 5 and 6, the control pad 17 portion of the housing 12 is accessible on the outer side of the housing 12 where control switches, lights, or connectors may be mounted for control of circuitry and/or audio devices that may include components positioned within the housing 12 and/or the ear pad coupled thereto.

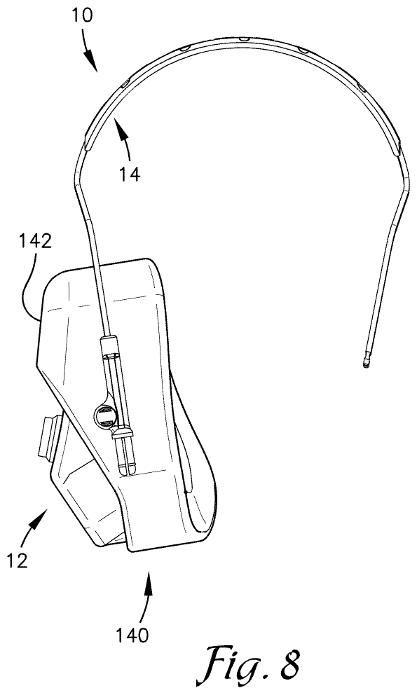

Turning to FIGS. 8 through 10, a headset 10, which includes a headband 14 and housing 12 as in FIG. 1, is depicted with a second embodiment of an exemplary ear pad 140 that includes an ear cover 142 attached to the housing 12. The headband 14 attaches to the housing 12 as previously described, with the arched headband 14 configured to extend upwardly around a wearer's head 101 to secure the headset 10 in place and position the housing 12 adjacent to the wearer's ear.

In this embodiment, the ear pad 140 and cover 142 are attached to and extend around at least a portion the upper portion 13 and the side wall 25 of the housing 12, which in turn is attached to the headband 14. As shown in the figures, the ear pad 140 extends outward from the side wall 25 and inward relative to the interior surface 26 of the housing 12 to extend completely or contiguously around the ear of the wearer. The ear pad 140, the ear cover 142 and the housing 12 form an earcup that rests around an outside of the ear and completely covers or encloses the ear to block a significant amount of ambient noise from reaching the ear. In this embodiment, the ear cover 142 is an outward extension of and/or formed integrally with the ear pad 140, and the cover 142 extends over and covers at least a portion of the housing 12. As described previously, the padding material of the ear pad 140 and ear cover 142 may be comprised of sound absorbing material to lessen the ambient noise level reaching the ear and may further be comprised of a comfortable padding material at the point(s) of contact of the pad with the wearer's head.

In this embodiment the ear cover 142 and ear pad 140 are joined to form a pocket or hollow with an exterior opening 146 into the pocket sized to allow insertion of the housing 12 therein and into which the housing 12 is inserted to secure the ear pad 140 and cover 142 to the housing 12. In alternative embodiments, the ear pad 140 and cover 142 may be attached to the housing by a releasable adhesive, by hook and loop fastener tabs, by an elastic band attached around the perimeter of the ear pad and/or cover that is stretched over the housing, or by any other releasable attachment means known in the art.

In this embodiment, the control pad 17 portion of the housing 12 is not covered by the cover 142 and is accessible on the outer side of the housing where control switches, lights, or connectors may be mounted for control of circuitry and/or audio devices that may be positioned within the housing 12 and/or ear pad 140 coupled thereto.

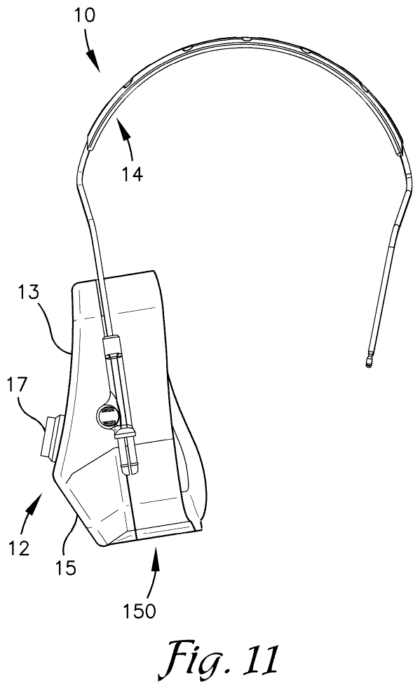

Looking to FIGS. 11 through 13, a headset 10, which includes a headband 14 and housing 12 as in FIG. 1, is depicted with a third embodiment of an exemplary ear pad 150 attached to the housing 12. The headband 14 attaches to the housing 12 as previously described, with the arched headband 14 configured to extend upwardly around a wearer's head 101 to secure the headset 10 in place and position the housing 12 adjacent to the wearer's ear.

In this embodiment, the ear pad 150 is attached to the housing 12, which in turn is attached to the headband 14. The ear pad 150 may be attached to the housing 12 by a releasable adhesive, by hook and loop fastener tabs, by an elastic band attached around the perimeter of the ear pad 150 so that the ear pad 150 is stretched over the housing 12, or by any other releasable attachment means known in the art.

As shown in the figures, the ear pad 150 in this embodiment is similar to that depicted in FIGS. 4 through 7, but the pad 150 in this embodiment extends completely around or is contiguous around the wearer's ear, with no gaps in the pad material to allow ambient sound to enter, minimizing or eliminating sound to the wearer's ear. In an embodiment, the ear pad 150 extends inward relative to the interior surface 26 of the housing 12. The ear pad 150 has a similar circumferential dimension to that of the housing 12, and the ear pad 150 and the housing 12 form an earcup that rests on the ear or rests around the outside of the ear. As described previously, the padding material of the ear pad 150 may be comprised of sound absorbing material to lessen the ambient noise level reaching the ear and may further be comprised of a comfortable padding material at the point(s) of contact of the pad with the wearer's head.

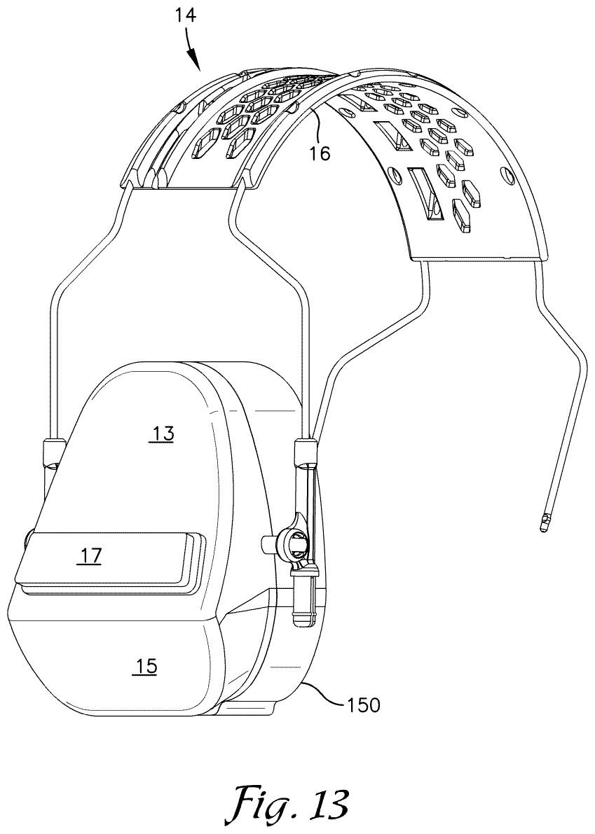

As best shown in FIG. 13, in this embodiment the housing 12 acts as the ear covering, covering the outer portion of the ear pad 150 to prevent, diminish or limit sound from directly entering the wearer's ear from that direction. In this embodiment, the control pad 17 portion of the housing 12 is accessible on the outer side of the housing where control switches, lights, or connectors may be mounted for control of circuitry and/or audio devices that may be positioned within the housing 12 and/or ear pad 150.

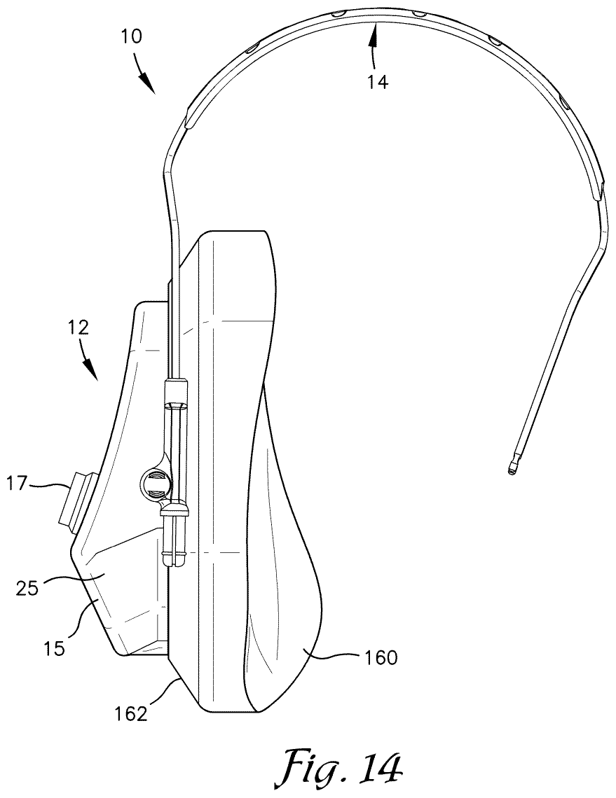

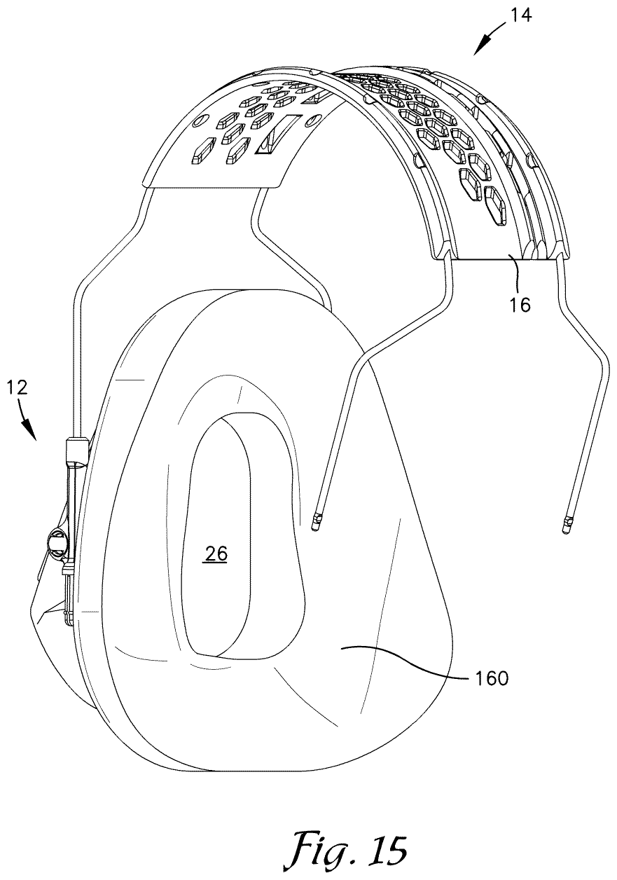

Turning to FIGS. 14 and 15, a headset 10, which includes a headband 14 and housing 12 as in FIG. 1, is depicted with a fourth embodiment of an exemplary ear pad 160 that includes an ear cover 162 attached to the housing 12. The headband 14 attaches to the housing 12 as previously described, with the arched headband 14 configured to extend upwardly around a wearer's head 101 to secure the headset 10 in place and position the housing 12 adjacent to the wearer's ear.

In this embodiment, the ear pad 160 and cover 162 are formed to fit and conform closely to the shape of the side of the wearer's head, i.e., an inner face or face engaging surface of the ear pad 160 is not flat, as may be provided in other embodiments, and is preferably contoured to conform to an average shape of a wearer's head surrounding the wearer's ear. It is also foreseen that the shape of the inner face of the ear pad 160 may be custom formed to conform to the intended wearer's head surrounding the wearer's ear. The ear pad 160 and cover 162 are attached to the housing 12, which in turn is attached to the headband 14. The ear pad 160 may be attached to the housing by a releasable adhesive, by hook and loop fastener tabs, by an elastic band attached around the perimeter of the ear pad and/or cover that is stretched over the housing, or by any other releasable attachment means known in the art.

As shown in the figures, the ear pad 160 with the cover 162 in this embodiment extends outward from the side wall 25 and inward relative to the interior surface 26 of the housing 12. The ear pad 160 extends completely or contiguously around the wearer's ear, with no gaps in the pad material to allow ambient sound to enter, minimizing or eliminating sound to the wearer's ear. In this embodiment, the ear cover 162 is an outward extension of and may be formed integrally with the ear pad 160, and the ear cover 162 is configured to extend outward from the side wall 25 of the housing 12 to form an earcup. The ear pad 160, the ear cover 162, and the housing 12 form the earcup that rests around an outside of the ear and completely covers or encloses the ear and forms a continuous or contiguous seal around the ear of the wearer. As described previously, the padding material of the ear pad 160 and/or ear cover 162 may be comprised of sound absorbing material to lessen the ambient noise level reaching the ear and may further be comprised of a comfortable padding material at the point(s) of contact of the pad with the wearer's head.

In this embodiment the housing 12 and the cover 162 act as the ear covering, covering the outer portion of the ear pad 160 to prevent, diminish or limit sound from directly entering the wearer's ear from that direction. In this embodiment, the control pad 17 portion of the housing 12 is accessible on the outer side of the housing where control switches, lights, or connectors may be mounted for control of circuitry and/or audio devices that may be positioned within the housing and/or ear pad.

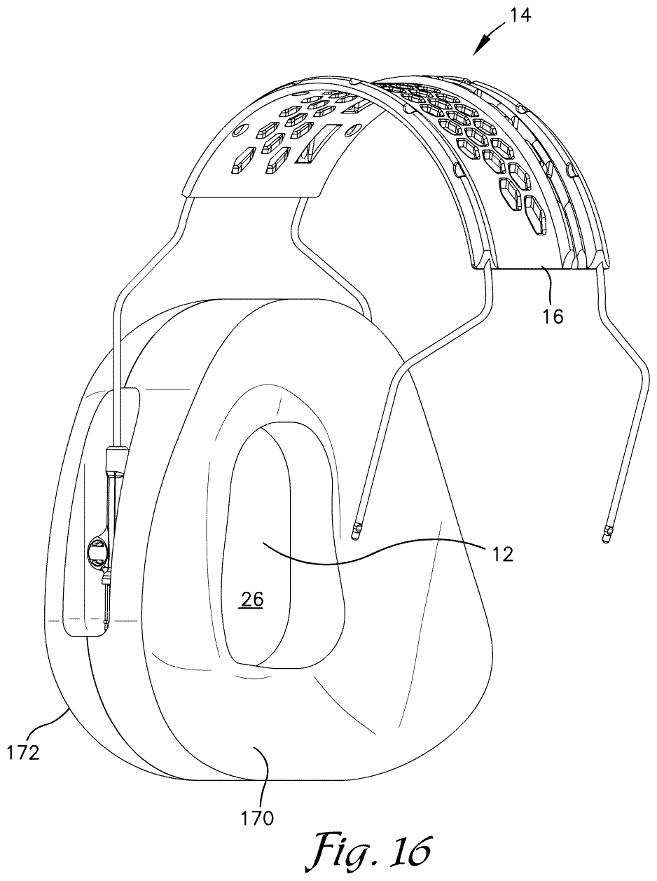

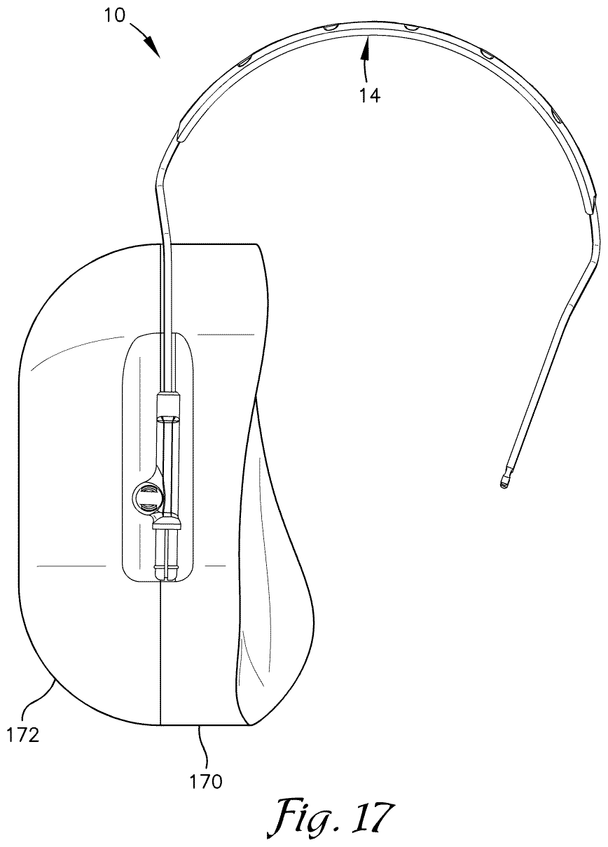

Looking to FIGS. 16 and 17, a headset 10, which includes a headband 14 and housing 12 as in FIG. 1, is depicted with a fifth embodiment of an exemplary ear pad 170 that includes an ear cover 172 attached to the housing 12. The headband 14 attaches to the housing 12 as previously described, with the arched headband 14 configured to extend upwardly around a wearer's head 101 to secure the headset 10 in place and position the housing 12 adjacent to the wearer's ear.

In this embodiment, the ear pad 170 and ear cover 172 are attached to and completely surround the housing 12, which in turn is attached to the headband 14. The ear pad 170 may be attached to the housing by a releasable adhesive, by hook and loop fastener tabs, by an elastic band attached around the perimeter of the ear pad so that the ear pad is stretched over the housing, or by any other releasable attachment means known in the art.

The ear pad 170 in this embodiment extends outward from the side wall 25 and inward relative to the interior surface 26 of the housing 12. The ear pad 170 extends completely around or is contiguous around the wearer's ear, with no gaps in the pad material to allow ambient sound to enter, minimizing or eliminating sound to the wearer's ear. And, in this embodiment, the ear cover 172 is an outward extension of the ear pad 170 and is preferably essentially contiguous with the ear pad 170 to surround the housing 12. The ear pad 170 and the ear cover 172 form an earcup that rests around an outside of the ear to completely cover or enclose the ear to block a significant amount of ambient noise from reaching the ear.

In an embodiment, the ear cover 172 and ear pad 170 are joined to form a pocket or hollow sized to allow insertion of the housing 12 therein and into which the housing 12 is inserted to secure the ear pad 170 and cover 172 to the housing 12 The padding material of the ear pad 170 and ear cover 172 may be comprised of sound absorbing material to lessen the ambient noise level reaching the ear, and may further be comprised of a comfortable padding material at the point(s) of contact of the pad with the wearer's head. The inner face of the ear pad 170 may be configured to correspond to an average shape of a wearer's head around the wearer's ear or may be custom shaped to conform to the shape of a specific wearer's head around the wearer's ear.

As shown in the figures, in this embodiment the ear cover 172 entirely covers the housing 12, so that the entirety of the ear pad and ear cover form a large sound absorbing barrier, with no gaps or hard surfaces to allow or conduct ambient noise.

As is also shown, in this embodiment the control pad of the housing is completely covered so that no gaps or spaces exist to allow incursion or entry of noise to the wearer, minimizing or eliminating sound to the wearer's ear.

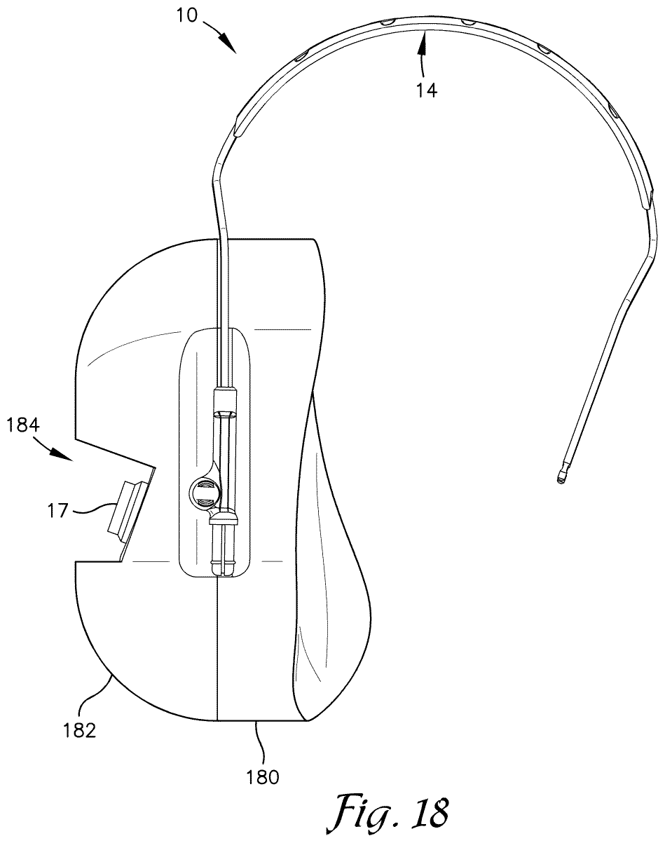

Looking to FIG. 18, a headset 10, which includes a headband 14 and housing 12 as in FIG. 1, is depicted with a sixth embodiment of an exemplary ear pad 180 that includes an ear cover 182 attached to the housing 12. The headband 14 attaches to the housing 12 as previously described, with the arched headband 14 configured to extend upwardly around a wearer's head 101 to secure the headset 10 in place and position the housing 12 adjacent to the wearer's ear.

In this embodiment, the ear pad 180 and ear cover 182 are attached to the housing 12, which in turn is attached to the headband 14. The ear pad 180 may be attached to the housing by a releasable adhesive, by hook and loop fastener tabs, by an elastic band attached around the perimeter of the ear pad so that the ear pad is stretched over the housing, or by any other releasable attachment means known in the art.

The ear pad 180 in this embodiment extends outward from the side wall 25 and inward relative to the interior surface 26 of the housing 12. The ear pad 180 extends completely around or is contiguous around the wearer's ear, with no gaps in the pad material to allow ambient sound to enter, minimizing or eliminating sound to the wearer's ear. And, in this embodiment, the ear cover 182 is an outward extension of the ear pad 180 and is preferably essentially contiguous with the ear pad 180 to almost completely surround the housing 12. The ear pad 180 and ear cover 182 form an earcup that rests around an outside of the ear to completely cover or enclose the ear to block a significant amount of ambient noise from reaching the ear. In an embodiment, the ear pad 180 and ear cover 182 are joined or integrally formed to form a pocket or hollow with an exterior opening 184 into the pocket sized to allow insertion of the housing 12 therein and into which the housing 12 is inserted to secure the ear pad 180 and ear cover 182 to the housing 12. The padding material of the ear pad 180 and ear cover 182 are preferably comprised of sound absorbing material to lessen the ambient noise level reaching the ear and may further be comprised of a comfortable padding material at the point(s) of contact of the pad with the wearer's head.

As shown in the figures, this embodiment of the ear pad 170 is similar to the embodiment shown in FIGS. 16 and 17, but the exterior opening or cutout 184 formed in the ear cover 172 allows access to the control pad 17 to allow user access to any switches or controls mounted on the control pad.

As shown in FIGS. 19 and 20, a headset 10, which includes a headband 14 and a first housing 12a as in FIG. 1, and further includes a second housing 12b, is depicted with a seventh embodiment of exemplary ear pads 190 attached to the housings 12a and 12b. Further, a communications system 200 is coupled to the headset 10. The first housing 12a is coupled between the tines 22a and 22b (not shown), and the second housing 12b coupled between the tines 24a and 24b. The headband 14 attaches to the housings 12a and 12b as previously described, with the arched headband configured to extend between the housings 12a and 12b to secure the headset 10 on a wearer's head 101 with the housings 12a and 12b positioned adjacent to the wearer's respective ears. FIG. 20 shows an embodiment of the second housing 12b coupled to the headset 10.

In this embodiment, the ear pads 190 are attached to the housings 12a and 12b, which in turn are attached to the headband 14. Each ear pad 190 may be attached to the respective housing 12a and 12b by a releasable adhesive, by hook and loop fastener tabs, by an elastic band attached around the perimeter of the ear pad so that the ear pad is stretched over the housing, or by any other releasable attachment means known in the art. In the embodiment shown, the ear pads 190 attached to the housings 12a and 12b are the same, but it is foreseeable that any combination of ear pads could be used in this embodiment.

The ear pads 190 in this embodiment are similar to the ear pad 150 shown in FIGS. 11 through 13 and are continuous around or contiguous around the wearer's ear, with no gaps in the pad material to allow ambient sound to enter, minimizing or eliminating sound to the wearer's ear. Further, the ear pads 190 have a similar circumferential dimension to that of the respective housing 12, such that each ear pad 190 and housing 12 form an earcup that rests on the ear or rests around the outside of the ear. As described previously, the padding material of the ear pads 190 is preferably comprised of sound absorbing material to lessen the ambient noise level reaching the ears and may further be comprised of a comfortable padding material at the point(s) of contact of the pads with the wearer's head.

As best shown in FIG. 20, the control pad 17 portion of the housing 12 is accessible on the outer side of the housing to control the communications system 200 positioned within the housing 12 and/or ear pad 150. The control pad 17 may include one or more buttons or sensors or switches that may be engaged by a user to control the communications system 200. The switches may have one or more functions. In an embodiment, a first switch may have a function of disconnecting power to the communications system 200 and/or a cancel/back function, and a second switch may have a function of providing power to the communications system 200 and/or a confirm/select function.

In the embodiment shown in FIGS. 19 and 20, the communications system 200 is coupled to the headset 10. The communications system 200 includes a microphone 202 coupled to a microphone cable 204 for capturing audio input from the wearer and a speaker (not shown) housed within a speaker housing 210 of each of the ear pads 190. The speaker housing 210 is positioned such that each speaker is positioned between the housing 12a or 12b and the respective ear pad 190. In an embodiment, the housings 12a and/or 12b may be a speaker housing in which the speaker and/or components associated therewith is housed. The speakers are coupled to an audio cable 212 for transmitting sound from an audio source to the wearer. The audio cable 212 extends through an audio cable channel 214 in the headband 14 to connect the speakers associated with each of the housings 12a and 12b to the audio source. In an embodiment shown in FIGS. 19 and 20, a chord 220 provides connection to a device for one or both of listening and speaking functionalities, including audio input, audio output, and/or a microphone input. The embodiment of the combined connecting chord 220 shown includes an audio jack known in the art, but other types of connectors may be used with the communications system 200, including, for example, a USB connector. In an embodiment, the communications system 200 is a wireless system that is connectable to a device via wireless technology or a wireless connection known in the art. In an embodiment, a battery port or another power supply 230 is coupled to or positioned within one of the housings 12a and/or 12b to provide power to the communications system 200.

Many different arrangements of the various components depicted, as well as components not shown, are possible without departing from the scope of the claims below. Embodiments of the technology have been described with the intent to be illustrative rather than restrictive. Alternative embodiments will become apparent to readers of this disclosure after and because of reading it. Alternative means of implementing the aforementioned can be completed without departing from the scope of the claims below. Identification of structures as being configured to perform a particular function in this disclosure and in the claims below is intended to be inclusive of structures and arrangements or designs thereof that are within the scope of this disclosure and readily identifiable by one of skill in the art and that can perform the particular function in a similar way. Certain features and sub-combinations are of utility and may be employed without reference to other features and sub-combinations and are contemplated within the scope of the claims.

1. A headset comprising:
a headband configured to extend around a portion of a head of a wearer; and
a first housing coupled to the headband and configured to be spaced from a first ear of the wearer; wherein,
the first housing is configured to receive a selected first ear pad releasably couplable thereto that provides a level of noise attenuation associated therewith, the selected first ear pad coupled to the first housing forming a first earcup, and
the headband is configured to position the first earcup relative to the first ear of the wearer.

2. The headset of claim 1, wherein the first housing is configured to receive each of a plurality of ear pads, each ear pad releasably couplable to the first housing to provide a level of noise attenuation associated therewith when coupled to the first housing and positioned relative to the first ear of the wearer.

3. The headset of claim 1, wherein the headband includes first and second support rods extending from at least one end of the headband, and the first and second support rods form respective first and second couplers that rotatably couple the first housing therebetween.

4. The headset of claim 1, wherein the headband comprises a band having one or more channels each having a support rod extending therethrough, and each support rod extends to engage the first housing.

5. The headset of claim 1, wherein the first housing includes a control pad for controlling components operatively coupled to the headset.

6. The headset of claim 1, further comprising a second housing coupled to the headband and configured to be spaced from a second ear of the wearer, wherein the second housing is configured to receive a selected second ear pad releasably couplable thereto that provides a level of noise attenuation associated therewith, the selected second ear pad coupled to the second housing forming a second earcup, and the headband is configured to position the second earcup relative to the second ear of the wearer.

7. A headset, comprising:
a headband configured to extend around a portion of a head of a wearer;
a first housing coupled to the headband; and
a plurality of ear pads each interchangeably couplable to the first housing to form an earcup configured to rest on or around an ear of the wearer; wherein,
each of the plurality of ear pads is configured to be selectable to provide a level of noise attenuation associated therewith, and
the headband positions the first housing such that each of the plurality of ear pads provides the level of noise attenuation associated therewith when coupled to the first housing.

8. The headset of claim 7, wherein the headband includes first and second support rods extending from the headband, and the first and second support rods form respective first and second couplers that rotatably couple the first housing therebetween.

9. The headset of claim 7, wherein the headband comprises a band having one or more channels each having a support rod extending therethrough, and each support rod extends to engage the first housing.

10. The headset of claim 7, wherein the first housing includes a control pad for controlling components operatively coupled to the headset.

11. The headset of claim 7, further comprising a second housing coupled to the headband, wherein the headband positions the second housing such that each of the plurality of ear pads provides the level of noise attenuation associated therewith when coupled to the second housing.

12. The headset of claim 7, wherein each of the plurality of ear pads comprises noise attenuation material to provide the level of noise attenuation associated therewith.

13. A headset, comprising:
a headband configured to extend around a portion of a head of a wearer;
a first housing coupled to a first end of the headband;
a second housing coupled to a second end of the headband; and
a plurality of ear pads each releasably couplable to the first housing and the second housing, and each of the plurality of ear pads providing a level of noise attenuation associated therewith; wherein,
the headband positions the first housing and the second housing relative to a first ear and a second ear of the wearer, respectively, to provide the level of noise attenuation associated with each of a first ear pad coupled to the first housing and a second ear pad coupled to the second housing, wherein the first ear pad and the second ear pad are selected from the plurality of ear pads.

14. The headset of claim 13, wherein the headband includes first and second support rods extending from the headband, and the first and second support rods form respective first and second couplers that rotatably couple the first housing and the second housing thereto.

15. The headset of claim 13, wherein the headband comprises a band having one or more channels each having a support rod extending therethrough, and each support rod extends to engage the first housing and the second housing.

16. The headset of claim 13, wherein at least one of the first housing and the second housing includes a control pad for controlling components operatively coupled to the headset.

17. The headset of claim 13, wherein each of the plurality of ear pads comprises noise attenuation material to provide the level of noise attenuation associated therewith.
